(19) United States
(12) Patent Application Publication
(10) Pub. No.: US 2026/0198847 A1
(43) Pub. Date: Jul. 16, 2026

(54) CLOSED-LOOP NEURAL STIMULATION FOR SLEEP APNEA TREATMENT

(71) Applicant: OpenMinds Lab, Inc., Rancho Santa Margarita, CA (US)

(72) Inventor: Haoxin Zhang, Irvine, CA (US)

(73) Assignee: OpenMinds Lab, Inc., Rancho Santa Margarita, CA (US)

(21) Appl. No.: 19/441,738

(22) Filed: Jan. 6, 2026

Related U.S. Application Data
(60) Provisional application No. 63/744,137, filed on Jan. 10, 2025.

(51) Int. Cl. A61B 5/00; A61B 5/0205; A61B 5/024; A61B 5/08; A61B 5/1455; A61B 5/346; A61B 5/372; A61B 5/397; A61N 1/04; A61N 1/36

(52) U.S. Cl. CPC A61B 5/4836; A61B 5/0205; A61B 5/02416; A61B 5/08; A61B 5/14553; A61B 5/346; A61B 5/372; A61B 5/397; A61B 5/4818; A61N 1/0456; A61N 1/0476; A61N 1/0492; A61N 1/36031; A61N 1/36034; A61B 2562/0204

(57) ABSTRACT

The present disclosure provides systems and methods for closed-loop neural stimulation to treat sleep apnea. A comprehensive monitoring system continuously tracks multiple physiological parameters to detect sleep states and apnea events. Based on this real-time data, a control module dynamically adjusts neural stimulation parameters delivered through a flexible electrode array system. The system supports multiple stimulation modes including single-pair amplitude-modulated stimulation and multi-pair configurations, allowing patient-specific optimization while minimizing discomfort.

### CROSS REFERENCE TO RELATED APPLICATIONS

This application claims priority under 35 U.S.C. 120 to Provisional U.S. Patent App. No. 63/744,137 (Atty Docket HXZGP001P) entitled “CLOSED-LOOP NEURAL STIMULATION FOR SLEEP APNEA TREATMENT”, filed Jan. 10, 2025, which is hereby incorporated by reference in its entirety and for all purposes.

### TECHNICAL FIELD

The present disclosure relates generally to systems for treating sleep apnea, and more particularly to closed-loop neural stimulation systems and techniques that adaptively control stimulation based on real-time physiological monitoring.

### BACKGROUND

Sleep apnea is a common sleep disorder characterized by repeated episodes of complete or partial upper airway obstruction during sleep. During an apnea event, the tongue muscles lose tone and relax, causing the tongue to slide backward and narrow the pharynx, leading to airway obstruction and interruption of normal breathing.

Current treatments for sleep apnea range from non-invasive therapies to surgical interventions. One of the most common treatments is Continuous Positive Airway Pressure (CPAP) therapy, which involves wearing a mask or nasal prongs that deliver a constant stream of air pressure into the airways, keeping them open during sleep. While CPAP is effective in reducing apnea events and improving oxygen saturation, it can be uncomfortable, noisy, and may cause dry mouth, nosebleeds, or skin irritation. Bi-Level Positive Airway Pressure (BiPAP) therapy is similar to CPAP but delivers two different levels of air pressure, a higher pressure during inhalation and a lower pressure during exhalation, which can be more comfortable for patients with severe sleep apnea.

For patients who have difficulty tolerating CPAP or BiPAP, oral appliances such as mandibular advancement devices may be an effective alternative. These custom-made devices advance the lower jaw forward, keeping the airway open during sleep, and are often used to treat mild to moderate sleep apnea. However, they may cause dental or temporomandibular joint (TMJ) issues and may not be suitable for patients with severe sleep apnea. Surgical treatments, such as uvulopalatopharyngoplasty (UPPP), maxillomandibular advancement (MMA), and tracheostomy, are typically reserved for patients with severe sleep apnea who have failed other treatments. These procedures can be effective in widening the airway or bypassing upper airway obstruction but carry risks associated with surgery, such as infection, bleeding, and scarring.

In recent years, hypoglossal nerve stimulation (HNS) therapy has emerged as a promising treatment for sleep apnea. This involves implanting a device that stimulates the hypoglossal nerve to keep the tongue muscles active during sleep, preventing airway obstruction. While HNS therapy can be effective for patients with moderate to severe sleep apnea who have failed other treatments, it requires surgical implantation and may cause side effects such as tongue numbness or pain. Consequently, it is desirable to provide continued improvements to systems and techniques for treatment of apnea or hypopnea that overcome at least some of the drawbacks of existing mechanisms.

### BRIEF DESCRIPTION OF THE DRAWINGS

FIG. 1 illustrates an example of a system for treatment of sleep apnea.

FIG. 2 illustrates an example of a monitoring module.

FIG. 3 illustrates an example of a control module.

FIG. 4 illustrates an example of a stimulation module.

FIG. 5 illustrates an example of a flow process diagram for treatment of sleep apnea or hypopnea.

FIG. 6 illustrates an example of a pre-modulated stimulation waveform.

FIG. 7 illustrates examples of electrode configurations for hypoglossal nerve stimulation.

FIG. 8 illustrates one example of a computing device.

### DETAILED DESCRIPTION

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

For example, the techniques of the present invention will be described in the context of sleep apnea and closed loop operation. However, it should be noted that the techniques of the present invention apply to a wide variety of different conditions as well as open loop operation. In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise. For example, parameters may include a variety of specific metrics. However, it will be appreciated that a system can use variety of different types of data while remaining within the scope of the present invention unless otherwise noted. Furthermore, the techniques and mechanisms of the present invention will sometimes describe a connection between two entities. It should be noted that a connection between two entities does not necessarily mean a direct, unimpeded connection, as a variety of other entities may reside between the two entities. For example, different layers may be connected using a variety of mechanisms. Consequently, a connection does not necessarily mean a direct, unimpeded connection unless otherwise noted.

Sleep apnea is a prevalent sleep disorder characterized by repeated episodes of complete or partial upper airway obstruction during sleep. During an apnea event, the tongue muscles lose tone and relax, causing the tongue to slide backward and narrow the pharynx, leading to airway obstruction and interruption of normal breathing. Traditional treatments have ranged from continuous positive airway pressure (CPAP) devices to surgical interventions, each with significant limitations in terms of patient comfort, adherence, and invasiveness.

Some neural stimulation techniques for treating conditions such as sleep apnea rely on direct electrical activation of peripheral nerves, including the hypoglossal nerve, to modulate muscle activity. In some approaches, surface or implanted electrodes deliver low-frequency pulsed waveforms (typically in the range of 1-100 Hz) that directly elicit action potentials in target nerves. However, these mechanisms often face limitations in selectively stimulating deep nerve structures without simultaneously activating overlying tissues, leading to discomfort, suboptimal therapeutic outcomes, or limited patient compliance.

To address these challenges, some techniques have explored non-invasive deep tissue stimulation using temporal interference (TI). Temporal interference stimulation involves the use of two or more high-frequency electrical signals, each above the excitation threshold of neurons (e.g., >1 kHz), delivered through separate electrode pairs. These signals differ slightly in frequency (e.g., 4000 Hz and 4050 Hz), resulting in a beat frequency (e.g., 50 Hz) created by their interference envelope. Neurons are insensitive to the high-frequency carrier waves but can respond to the amplitude-modulated low-frequency envelope where the electric fields spatially overlap. This allows for selective stimulation of deep neural targets while minimizing activation of superficial nerves and skin sensory receptors.

In some TI implementations, electrode placement shapes the intersecting electric fields. A typical configuration employs a square arrangement with two pairs of electrodes placed at opposite vertices. Each pair delivers one of the high-frequency signals, and the region of field overlap where the interference envelope is sufficient to depolarize neurons, is determined by the relative positioning, amplitude, and frequency of the signals. This setup allows focusing of stimulation at depth, but it requires precise alignment and often a larger electrode footprint, which may not be suitable for all anatomical configurations, especially in patients with smaller jaw structures.

According to various embodiments, mechanisms are provided for adaptive neural stimulation to treat sleep apnea that overcome the limitations of other approaches. The disclosed system recognizes that temporal interference is not a physiological requirement for effective nerve stimulation, but rather one possible implementation strategy among several alternatives. In various embodiments, the system provides flexible stimulation regimes including: a simplified one-pair configuration using amplitude-modulated high-frequency carriers that eliminates the need for temporal interference while maintaining the benefits of reduced surface sensation, a two-pair temporal interference configuration for cases where enhanced spatial focusing is beneficial, and a three-pair or more configuration that provides additional degrees of freedom for field shaping and improved selectivity. Various configurations integrate with a closed-loop monitoring and control system that continuously adapts stimulation parameters based on real-time physiological feedback, allowing patient-specific optimization and customization that is absent from typical open-loop approaches.

Many systems also rely on open-loop stimulation where stimulation is delivered according to a fixed schedule or preset parameters regardless of the patient's current physiological state. For example, the device may activate at bedtime and deliver continuous or periodic stimulation throughout the night without monitoring whether apnea events are actually occurring. While simple in design, this approach risks over-stimulation during periods of stable breathing, leading to unnecessary discomfort, accelerated tissue desensitization (tolerance), and reduced battery life. Moreover, it may fail to respond appropriately to transient or variable airway obstructions that require immediate, on-demand intervention.

In contrast, various embodiments provide an intelligent closed-loop system that operates based on real-time physiological feedback. The system continuously monitors key biomarkers such as respiratory effort, airflow, blood oxygen saturation, snoring intensity, and/or electromyographic (EMG) activity of upper airway muscles. When early signs of airway collapse or an apnea event are detected, the control module triggers or adjusts stimulation parameters (e.g., amplitude, frequency, pulse width, or electrode configuration) to restore patency. After normalization of breathing, stimulation can be reduced or paused, minimizing side effects.

According to various embodiments, the integration of closed-loop control significantly enhances the therapeutic precision of neural stimulation. For instance, during transitions between sleep stages, when upper airway muscle tone naturally fluctuates, the system can proactively and often preventatively modulate stimulation intensity to maintain airway stability. This adaptability is particularly important because sleep apnea events vary in frequency, duration, and severity both within and across nights.

Furthermore, the closed-loop architecture supports personalized treatment by learning individual response patterns over time. In advanced embodiments, machine learning algorithms analyze historical data to predict apnea onset and optimize stimulation timing and location. This level of responsiveness is unattainable with open-loop systems.

While open-loop devices are easier to implement and have been used in earlier generations of nerve stimulators (e.g., some hypoglossal nerve implants), they lack the dynamic responsiveness needed for optimal patient outcomes. In various embodiments, the present system advances the state of the art by combining adaptive, multi-electrode stimulation, including both TI-based and non-TI regimes, with closed-loop control, allowing efficient, comfortable, and physiologically aligned treatment of sleep apnea.

In various embodiments, the system operates using a two-pair (2-pair) regime consistent with traditional TI principles. Two electrode pairs deliver high-frequency biphasic sinusoidal waveforms, such as 4000 Hz and 4050 Hz, spatially oriented in a trapezoidal or approximately square pattern. The interference between these signals generates a low-frequency envelope (e.g., 50 Hz) within the target tissue, allowing focused stimulation of the hypoglossal nerve without direct activation of surrounding tissues. In some embodiments, the low-frequency envelope corresponds to a desired hypoglossal nerve neural activation rhythm of 20-50 Hz.

In contrast, various embodiments employ a one-pair (1-pair) regime that achieves effective neural stimulation without requiring temporal interference between multiple electrode pairs. In this mode, a single pair of electrodes (comprising an anode and cathode) delivers a pre-modulated biphasic waveform in which a high-frequency carrier (e.g., 1500-10000 Hz, and in particular embodiments greater than 2000 Hz, 3000 Hz, or 4000 Hz) is amplitude-modulated by a lower-frequency envelope (e.g., 0-500 Hz, more particularly 10-200 Hz, and in specific implementations 20-100 Hz). The high-frequency carrier serves multiple functions: it reduces skin impedance through capacitive coupling, minimizes uncomfortable surface sensations that occur with low-frequency direct stimulation, and allows charge-balanced stimulation with reduced electrochemical reactions at the electrode-tissue interface. The low-frequency envelope, which is the component to which nerve membranes physiologically respond, is directly encoded in the amplitude modulation and does not rely on beat frequency generation from multiple sources. This approach produces a physiologically equivalent effect to TI in terms of nerve activation. The nerve responds to the low-frequency modulation envelope while remaining largely insensitive to the high-frequency carrier. However, this eliminates the hardware complexity, electrode footprint, and precise frequency-matching requirements of multi-pair TI systems. The 1-pair regime is particularly beneficial for individuals with smaller anatomies (smaller jaw structures, thinner neck profiles) where placement of multiple electrode pairs may be geometrically impractical, uncomfortable, or would result in suboptimal field distributions, and for applications where device simplicity, cost reduction, and ease of use are priorities.

Additionally, alternative embodiments include a three-pair (3-pair) regime, where multiple electrode pairs (three or more) are independently controlled to shape and focus the electric field distribution with enhanced precision. By adjusting the relative phases, amplitudes, frequencies, and timing across three or more pairs, the system can generate more complex interference patterns, improve spatial selectivity (reducing activation of off-target tissues), and enhance stimulation efficiency particularly in anatomically complex regions where the hypoglossal nerve path varies significantly between individuals or where surrounding tissue conductivity is heterogeneous.

Electrode patches in various implementations maintain a flexible, conformable design to ensure reliable skin contact and consistent performance during sleep. In the 1-pair configuration, the electrode pair is aligned along the anatomical path of the hypoglossal nerve. In the 2-pair trapezoidal configuration, each pair is positioned at opposing points to maximize field overlap at depth. The system dynamically selects active electrodes and adjusts stimulation parameters, such as frequency, amplitude, and modulation depth, based on real-time physiological feedback in a closed-loop manner, thereby optimizing therapeutic efficacy while minimizing side effects. A 3-pair approach further tunes stimulation for particular patients.

These various embodiments collectively represent a flexible, patient-adaptive neurostimulation platform that encompasses temporal interference-based mechanisms, novel single-pair amplitude-modulated strategies that eliminate TI requirements, and advanced multi-pair configurations for enhanced field shaping. This diversity of approaches, combined with closed-loop physiological monitoring and machine learning-based optimization, offers improved clinical outcomes through enhanced patient comfort, broader anatomical compatibility, reduced device complexity and cost, and superior therapeutic efficacy compared to fixed-parameter or open-loop alternatives.

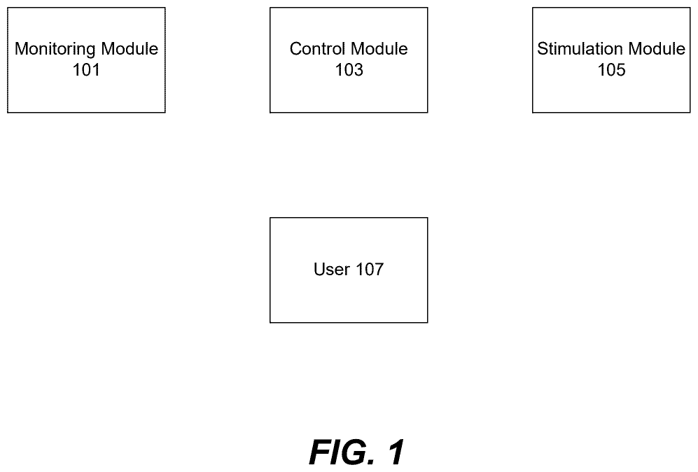

FIG. 1 illustrates an example of a system for treatment of sleep apnea. The system 100 includes a monitoring module 101, a control module 103, a stimulation module 105, and a user 107, configured in a closed-loop architecture to detect and treat sleep apnea events in real-time.

According to various embodiments, monitoring module 101 includes a plurality of physiological sensors configured to continuously track one or more physiological parameters associated with the user 107. These parameters may include, but are not limited to, respiratory effort, airflow, blood oxygen saturation, snoring intensity, electromyography (EMG) activity of upper airway muscles, electroencephalogram (EEG) signals, heart rate via electrocardiogram (ECG) or photoplethysmography (PPG), functional near-infrared spectroscopy (fNIRS), and audio signals from a neck-mounted or ambient microphone. The monitoring module may operate in real time, sampling data at a high frequency (e.g., ≥100 Hz) to allow rapid detection of transient respiratory events.

The control module 103 receives processed sensor data from the monitoring module 101 and dynamically adjusts stimulation parameters based on real-time physiological feedback. In particular embodiments, the control module 103 comprises a processor configured to detect apnea events associated with the user 107 using the plurality of physiological parameters. According to various embodiments, the control module 103 implements advanced machine learning algorithms to optimize stimulation patterns based on patient response. The control algorithms can be customized using patient-specific medical imaging data such as MRI or CT scans to account for individual anatomical variations. In some examples, the control module 103 operates with a primary control loop running at approximately 1 kHz to maintain precise timing of stimulation waveforms, and a secondary loop running at approximately 512 Hz to handle parameter updates based on sensor data.

The stimulation module 105 is configured to deliver electrical stimulation to the hypoglossal nerve of the user 107 in response to detection of apnea or hypopnea events. In particular embodiments, the stimulation module 105 includes a flexible electrode array that can be configured in various operational modes including a one-pair mode, a two-pair mode, and a three-pair mode. According to various embodiments, in the one-pair mode, a single pair of electrodes aligned longitudinally with the hypoglossal nerve delivers a charge-balanced biphasic waveform with a high-frequency sine wave or high-frequency carrier wave above 1500 Hz that is amplitude modulated by a low-frequency envelope in the range of 0-500 Hz. In the two-pair mode, two electrode pairs arranged in a trapezoidal or other geometric configuration deliver high-frequency biphasic waveforms at slightly different frequencies to create a temporal interference pattern at the hypoglossal nerve. In the three-pair mode, three or more electrode pairs provide additional spatial degrees of freedom for enhanced field shaping and improved selectivity. In some examples, the control module 103 automatically selects among these operational modes based on factors including user anatomy, detected apnea severity, electrode-tissue impedance measurements, and therapeutic efficacy metrics.

According to various embodiments, the system operates in a closed-loop configuration where the stimulation module 105 continues to stimulate the hypoglossal nerve until apnea events are no longer detected by the monitoring module 101 and the control module 103. In particular implementations, real-time feedback from the monitoring module 101 allows continuous optimization of stimulation parameters including parameter selected from the group consisting of amplitude, frequency, pulse width, electrode configuration, modulation depth, and duty cycle to maximize therapeutic efficacy while minimizing energy consumption and potential side effects. In some examples, the system 100 logs comprehensive data about sleep patterns, apnea events, and stimulation responses, which undergoes automated analysis to generate clinical reports and track treatment efficacy over time.

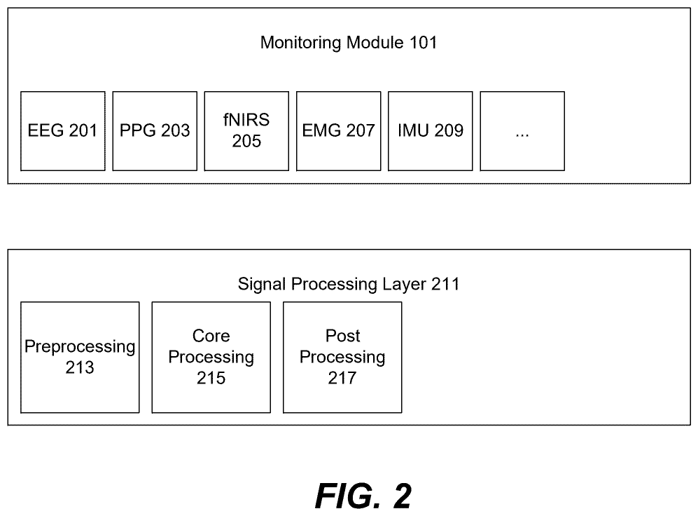

FIG. 2 illustrates an example of a monitoring module 101 for tracking physiological parameters associated with sleep apnea detection and treatment. The monitoring module 101 includes physiological sensors and a signal processing layer 211 configured to analyze and process sensor data through multiple algorithmic stages.

According to particular embodiments, the monitoring module 101 may incorporate multiple sensor types to provide comprehensive physiological monitoring. An EEG sensor 201 is configured to monitor brain electrical activity associated with sleep stages, arousal, sleep efficiency, and sleep latency, and may be particularly useful in initially configuring and customizing a monitoring module 101 for the user. In some examples, a PPG sensor 203 is configured to measure blood oxygen levels and pulse characteristics including heart rate, pulse wave amplitude, and respiratory-induced variations in the photoplethysmography signal. This PPG sensor 203 may be a separate sensor or may be included in a smartwatch or smartband. In particular embodiments, an fNIRS sensor 205 is configured to assess tissue oxygenation in the prefrontal cortex or other brain regions, providing complementary information about cerebral blood flow and oxygenation status. The EMG sensor 207 is configured to detect muscle activity including chin EMG for detecting REM sleep and muscle tone changes associated with apnea events. This may be incorporated into a strap configured to hold electrodes in some examples. The IMU 209, which may include accelerometers and gyroscopes, is configured to track body position, head orientation, and movement patterns during sleep. According to various embodiments, additional sensors (represented by the ellipsis) may include ECG sensors for heart rate monitoring, audio sensors to monitor breathing sounds and snoring, cameras, temperature sensors, and frequency modulated continuous wave (FMCW) radar sensors for contactless breathing monitoring.

According to various embodiments, the signal processing layer 211 receives raw sensor data from the multiple physiological sensors and processes the data through three sequential stages to extract relevant features and detect apnea events. In particular embodiments, the preprocessing component 213 performs signal conditioning, artifact removal, and noise reduction on the raw sensor data. According to various embodiments, the preprocessing component 213 implements adaptive filtering to remove motion artifacts and electrical noise from the sensor signals. Raw sensor data undergoes band-pass filtering specific to each sensor type, for example, EEG signals may be filtered in the 0.5 Hz to 60 Hz range to capture relevant sleep oscillations, while EMG signals may use a 10-500 Hz range to detect muscle activity. In some examples, the preprocessing component 213 employs independent component analysis (ICA) to separate artifact components from physiological signals.

In various implementations, the core processing component 215 receives preprocessed sensor data and extracts relevant features to detect potential apnea events using advanced signal processing techniques. In particular embodiments, the core processing component 215 uses a multi-modal fusion approach to detect apnea events by analyzing signals from multiple sensors simultaneously. According to various embodiments, PPG signals are analyzed to extract features related to tissue oxygen saturation, blood oxygen saturation, pulse wave characteristics, and respiratory rhythm. The core processing component 215 may calculate heart rate variability (HRV) metrics from ECG signals as additional markers of autonomic nervous system response during apnea events. Motion data from accelerometers and gyroscopes can undergo quaternion-based processing to track head position and respiratory effort. Audio data from audio sensors and microphones can be used to monitor breathing sounds and detect snoring patterns indicative of airway obstruction. In some examples, the core processing component 215 identifies patterns in the sensor data that are characteristic of apnea onset, including oxygen desaturation, changes in heart rate variability, increases in respiratory effort, and alterations in sleep stage architecture.

The post-processing component 217 receives extracted features from the core processing component 215 and applies machine learning algorithms to classify events and analyze sleep patterns. According to various embodiments, the post-processing component 217 implements machine learning models trained on historical physiological data to distinguish between normal breathing, hypopnea events, and apnea events. In particular embodiments, these machine learning algorithms may be applied for a particular user to allow patient-specific pattern recognition, for a particular category of individuals sharing similar characteristics such as age, gender, body mass index, or apnea severity, or can be applied broadly to the general user base. In some examples, the post-processing component 217 continuously updates event classifications based on accumulating data within a sleep session and provides confidence metrics for detected events. The output of the signal processing layer 211 is transmitted to the control module for determination of appropriate stimulation parameters and timing.

According to various embodiments, the monitoring module 101 maintains a rolling buffer of sensor data, for example a 5-minute buffer, to detect trends and patterns in breathing disturbances over time. This temporal context allows the system to distinguish isolated breathing irregularities from sustained apnea patterns requiring intervention. In particular implementations, the monitoring module 101 operates continuously throughout the sleep period to provide real-time feedback for closed-loop control of the stimulation module.

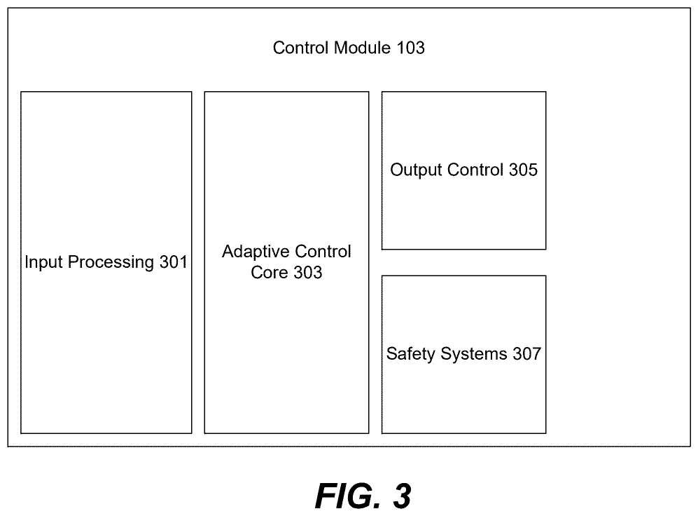

FIG. 3 illustrates an example of a control module 103 for managing closed-loop neural stimulation in sleep apnea treatment. According to various embodiments, the control module 103 includes an input processing component 301, an adaptive control core 303, an output control component 305, and safety systems 307, which together allow dynamic adjustment of stimulation parameters based on real-time physiological feedback.

In particular embodiments, the input processing component 301 receives processed sensor data from the monitoring module and prepares it for analysis and decision-making. According to various embodiments, the input processing component 301 receives physiological parameters including sleep stage information, respiratory event classifications, oxygen saturation levels, heart rate variability metrics, muscle activity measurements, and body position data. The input processing component 301 may perform additional data fusion and feature extraction to create a comprehensive representation of the patient's current physiological state. In some examples, the input processing component 301 normalizes and scales the incoming data streams to allow effective processing by the adaptive control core 303. According to various embodiments, the input processing component 301 also receives electrode-tissue impedance measurements, stimulation history, and patient comfort metrics to inform subsequent control decisions.

According to various implementations, the adaptive control core 303 analyzes the processed input data and determines optimal stimulation strategies for the current physiological state. The adaptive control core 303 implements one or more adaptive control algorithms configured to iteratively modify stimulation parameters based on real-time physiological feedback and historical response data. Such algorithms may include learning-based, optimization-based, rule-based, or hybrid approaches that improve therapeutic efficacy over time. In particular embodiments, the adaptive control core 303 implements advanced machine learning algorithms that learn and improve stimulation patterns based on patient response over time. According to various embodiments, the adaptive control core 303 evaluates stimulation effectiveness using one or more objective functions or performance metrics that combine multiple physiological and therapeutic criteria. In some examples, the adaptive control core 303 maintains or updates a patient-specific response model that maps stimulation parameters to observed physiological outcomes. The adaptive control core 303 may implement algorithms such as reinforcement learning, Q-learning, policy gradient methods, or actor-critic architectures to continuously refine the stimulation strategy. In particular embodiments, the adaptive control core 303 may predict apnea onset based on learned physiological patterns, allowing proactive rather than purely reactive stimulation.

The output control component 305 translates the decisions from the adaptive control core 303 into specific stimulation parameters for delivery by the stimulation module. According to various embodiments, the output control component 305 determines which electrodes to activate, the stimulation frequency, amplitude, pulse width, modulation depth, duty cycle, and waveform characteristics. In particular embodiments, the output control component 305 selects among operational modes including the one-pair regime, two-pair regime, or three-pair regime based on factors such as user anatomy, detected apnea severity, and therapeutic efficacy. The output control component 305 may implement current steering algorithms to shape the electric field distribution along the hypoglossal nerve by adjusting the relative phases, amplitudes, and frequencies across multiple electrode pairs. In some examples, the output control component 305 operates at a high update frequency, such as 512 Hz, to allow rapid parameter adjustments in response to changing physiological conditions. According to various embodiments, the output control component 305 ensures smooth transitions between stimulation states to avoid abrupt changes that could cause discomfort or arousal from sleep.

The safety systems 307 monitor all control decisions and stimulation outputs to ensure patient safety throughout treatment. In particular embodiments, the safety systems 307 enforce hard limits on stimulation parameters including maximum current density, maximum voltage, maximum duty cycle, and maximum stimulation duration to prevent tissue damage or excessive discomfort. According to various embodiments, the safety systems 307 continuously monitor electrode-tissue impedance and can pause or terminate stimulation if impedance values fall outside acceptable ranges, which may indicate poor electrode contact or skin irritation. The safety systems 307 may implement fault detection algorithms that identify anomalous sensor readings, control system failures, or unexpected physiological responses. In some examples, the safety systems 307 maintain a watchdog timer that requires periodic confirmation from the adaptive control core 303 and output control component 305, automatically disabling stimulation if the control system becomes unresponsive. According to various embodiments, the safety systems 307 ensure charge balance in all stimulation waveforms to prevent electrochemical tissue damage and maintain compliance with electrical safety standards for medical devices.

In some embodiments, the control module 103 also implements comprehensive data logging functionality that records sleep patterns, detected apnea events, applied stimulation parameters, and patient responses throughout each treatment session. This logged data undergoes automated analysis to generate clinical reports and track treatment efficacy over time, allowing clinicians to assess therapeutic outcomes and adjust treatment protocols as needed. According to various embodiments, the control module 103 can securely transmit anonymized data to a cloud platform for population-level analysis and algorithm improvement, contributing to ongoing refinement of the machine learning models and benefiting the broader user population through shared learning from aggregated treatment data

In particular implementations, the control module 103 operates with a primary control loop running at approximately 1 kHz to maintain precise timing of stimulation waveforms coordinated with the output control component 305. According to various embodiments, the control module 103 can incorporate patient-specific medical imaging data such as magnetic resonance imaging (MRI) or computed tomography (CT) scans during the adaptive control process to account for individual anatomical variations in nerve location, tissue conductivity, and optimal electrode placement. In some examples, during initial setup, the control module 103 performs a calibration sequence to map the relationship between stimulation parameters and tongue muscle activation, creating the patient-specific response model that is maintained and refined by the adaptive control core 303 over subsequent treatment sessions.

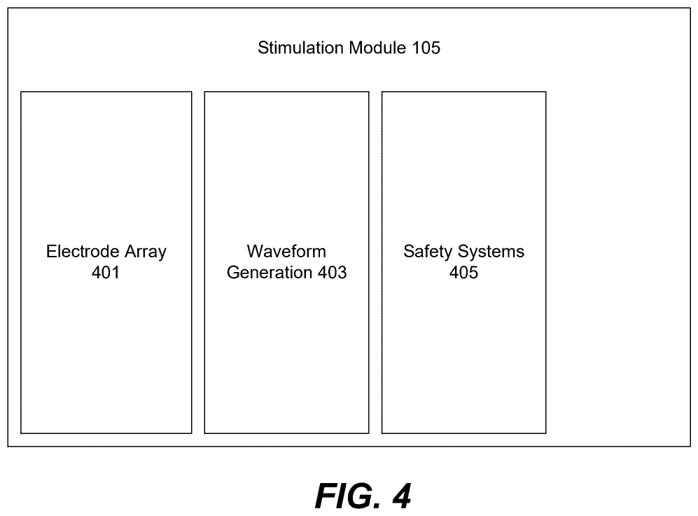

According to various embodiments, FIG. 4 illustrates an example of a stimulation module 105 for delivering neural stimulation to treat sleep apnea. The stimulation module 105 includes an electrode array 401, a waveform generation component 403, and a safety system 405, which together enable precise and safe delivery of electrical stimulation to the hypoglossal nerve.

In particular embodiments, the electrode array 401 comprises a flexible array of electrodes that can be configured in various arrangements to enhance stimulation coverage and accommodate different patient anatomies. According to various embodiments, the electrode array 401 may be configured in multiple operational modes including a one-pair mode, a two-pair mode, and a three-pair mode. In the one-pair mode, a single pair of electrodes comprising an anode and cathode is aligned longitudinally with the anatomical path of the hypoglossal nerve to maximize the longitudinal field gradient and provide selective recruitment with the lowest activation threshold. This configuration is particularly beneficial for individuals with smaller jaw structures or thinner neck profiles where placement of multiple electrode pairs may be geometrically impractical or uncomfortable. In the two-pair mode, two electrode pairs are arranged in a trapezoidal or other geometric configuration with each pair positioned at opposing points to maximize electric field overlap at depth, allowing temporal interference-based stimulation when desired. In the three-pair mode, three or more electrode pairs provide additional spatial degrees of freedom for enhanced field shaping, improved selectivity, and the ability to steer current through complex anatomical regions. According to various embodiments, the electrode array 401 may be configurable in larger arrays to provide even greater flexibility in field shaping and electrode selection.

The electrode array 401 may be integrated into a patient-specific electrode patch that conforms to the user's neck and jawline contours, ensuring comfortable and secure fit as well as accurate patient-specific electrode placement to enhance stimulation of the hypoglossal nerve. According to various embodiments, the electrode placement within the patch can be customized based on finite element modeling (FEM) simulation derived from imaging data such as MRI or CT scans of the user's neck and jaw region. The patch may be created using a customized mold generated from a 3D model of the patient's anatomy, which is then used to fabricate or 3D print the patch with electrodes positioned at optimal locations for that individual patient. In some examples, the electrode array 401 incorporates impedance monitoring circuits that continuously measure electrode-tissue interface conditions, allowing the control module to adapt stimulation parameters to maintain consistent current delivery despite variations in skin hydration, electrode contact pressure, or other factors. The electrodes may use a novel hydrogel interface material that maintains low impedance while allowing long-term wear comfort throughout the night.

According to various embodiments, the waveform generation component 403 receives stimulation parameters from the control module and generates precisely controlled electrical waveforms for delivery through the electrode array 401. In particular embodiments, the waveform generation component 403 uses direct digital synthesis (DDS) to maintain accurate frequency relationships between electrode pairs and enable rapid parameter adjustments. According to various embodiments, in the one-pair operational mode, the waveform generation component 403 generates a pre-modulated biphasic waveform in which a high-frequency carrier wave, typically above 1500 Hz and in particular embodiments greater than 2000 Hz, 3000 Hz, or 4000 Hz, is amplitude-modulated by a low-frequency envelope in the range of 0-500 Hz, more particularly 10-200 Hz, and in specific implementations 20-100 Hz. The high-frequency carrier reduces skin impedance through capacitive coupling and minimizes uncomfortable surface sensations, while the low-frequency envelope provides the neural activation signal to which the hypoglossal nerve fibers respond. In the two-pair operational mode, the waveform generation component 403 generates high-frequency biphasic sinusoidal waveforms for each electrode pair, for example in the ranges of 4000-4160 Hz or 10000-10160 Hz. According to various embodiments, pairs of electrodes operate at slightly different frequencies, such as one pair delivering a 4000 Hz signal while another delivers 4050 Hz, creating a temporal interference pattern with a difference frequency of 50 Hz in the target tissue. In the three-pair operational mode, the waveform generation component 403 independently controls the phase, amplitude, and frequency of signals delivered by each pair to shape the electric field distribution along the hypoglossal nerve with enhanced precision.

In particular embodiments, the waveform generation component 403 ensures that all generated waveforms are charge-balanced biphasic waveforms to prevent electrochemical tissue damage and maintain safe operation. According to various embodiments, the waveform generation component 403 can dynamically adjust stimulation parameters including amplitude, frequency, pulse width, modulation depth, and duty cycle in response to control signals received from the control module at update rates of 512 Hz or higher. In some examples, the waveform generation component 403 implements smooth ramping of amplitude changes to avoid abrupt stimulation transitions that could cause patient arousal or discomfort. In particular embodiments, the control module 103 makes determinations on whether to initiate stimulation and whether to adjust stimulation parameters at a frequency of approximately 10 Hz, using numerous parameters from the monitoring module including current physiological state, recent event history, and electrode-tissue interface conditions.

The safety system 405 monitors all aspects of stimulation delivery to ensure patient safety and device reliability. In particular embodiments, the safety system 405 enforces hard limits on stimulation parameters including maximum current amplitude, maximum voltage, maximum charge per phase, and maximum duty cycle to prevent tissue damage, burns, or excessive patient discomfort. According to various embodiments, the safety system 405 continuously monitors the electrode-tissue impedance measurements provided by the impedance monitoring circuits in the electrode array 401 and can automatically pause or terminate stimulation if impedance values indicate poor electrode contact, excessive skin dryness, or potential electrode degradation. The safety system 405 may verify charge balance for each stimulation pulse to ensure that the net charge delivered to tissue over each cycle is zero or within acceptable tolerances. In some examples, the safety system 405 implements thermal monitoring to detect excessive heating at the electrode-tissue interface and can reduce stimulation intensity or pause delivery if temperature thresholds are exceeded. According to various embodiments, the safety system 405 includes redundant monitoring pathways and fail-safe mechanisms that disable stimulation output if control system errors, sensor failures, or communication failures are detected. The safety system 405 may also log all stimulation events, safety limit violations, and system faults for subsequent clinical review and device troubleshooting.

According to various embodiments, the stimulation module 105 can deliver stimulation to either one or both hypoglossal nerves depending on the detected apnea pattern and patient anatomy. In particular embodiments, the stimulation module 105 continues to deliver stimulation until apnea events are no longer detected by the monitoring module and control module, allowing closed-loop operation that responds dynamically to the patient's changing physiological state throughout the night.

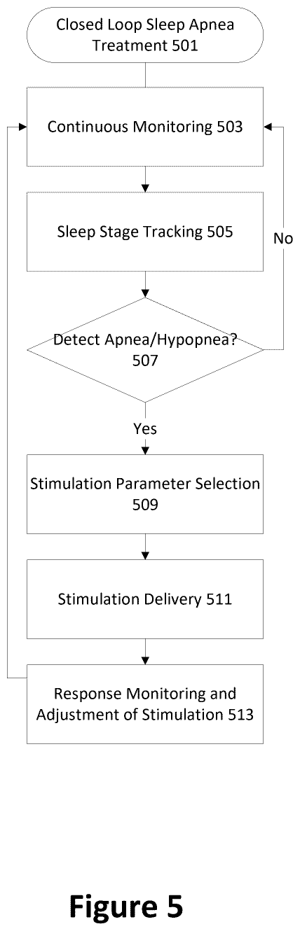

FIG. 5 illustrates an example flow process diagram for closed-loop sleep apnea treatment 501. At 503, the monitoring module continuously tracks multiple physiological parameters associated with the user throughout the sleep period. In particular embodiments, the continuous monitoring 503 involves acquiring data from a plurality of physiological sensors including EEG sensors, PPG sensors, fNIRS sensors, ECG sensors, EMG sensors, accelerometers, gyroscopes, audio sensors, and other sensing modalities. According to various embodiments, the sensor data is continuously processed through preprocessing, core processing, and post-processing stages to extract relevant features related to sleep state, respiratory patterns, oxygen saturation, cardiac activity, and other physiological markers. The continuous monitoring 503 operates throughout the entire treatment session, providing an ongoing stream of physiological data to inform subsequent decision-making steps.

According to various embodiments, the process proceeds to sleep stage tracking at 505, where the system analyzes EEG signals, PPG signals, and other sensor data to determine the user's current sleep stage. In particular embodiments, the sleep stage tracking 505 classifies sleep into stages such as wake, light sleep (N1, N2), deep sleep (N3), and REM sleep. According to various embodiments, sleep stage information is important because the propensity for apnea events, the degree of upper airway muscle tone, and the appropriate stimulation parameters may vary across different sleep stages. In some examples, the system maintains a rolling history of sleep stage transitions to provide temporal context for apnea detection and treatment decisions. The sleep stage tracking 505 allows the system to adapt its monitoring sensitivity and stimulation strategies based on the physiological characteristics of each sleep stage.

In particular embodiments, the control module evaluates the processed physiological data at decision point or apnea detection decision point 507 to determine whether an apnea or hypopnea event is occurring or is imminent. In particular embodiments, apnea detection 507 may involve identifying complete cessation of airflow for a specified duration (typically 10 seconds or more), while hypopnea detection involves identifying partial reduction in airflow accompanied by oxygen desaturation or arousal. According to various embodiments, the detection 507 may also incorporate predictive capabilities where a machine learning model analyzes subtle changes in physiological patterns to predict high probability of a respiratory event occurring soon, allowing proactive intervention before complete airway collapse occurs. In some examples, the detection algorithms fuse information from multiple sensor modalities including respiratory effort signals, oxygen saturation trends, heart rate variability changes, body position, and breathing sounds to improve detection accuracy and reduce false positives.

If no apnea or hypopnea is detected at decision point 507, the process returns to continuous monitoring 503, forming a monitoring loop that continuously assesses the user's physiological state without delivering unnecessary stimulation. This pathway represents periods of stable breathing where therapeutic intervention is not required. According to various embodiments, this conditional stimulation approach minimizes patient exposure to electrical stimulation during normal breathing, thereby reducing the risk of tissue desensitization, patient discomfort, and unnecessary energy consumption.

If an apnea or hypopnea event is detected (or predicted) at decision point 507, the process proceeds to stimulation parameter selection at 509. In particular embodiments, the stimulation parameter selection 509 involves the control module, including the adaptive control core, determining the optimal stimulation strategy for the current physiological state and detected event characteristics. According to various embodiments, the parameter selection at 509 includes determining which electrodes to activate from the electrode array based on the operational mode (one-pair, two-pair, or three-pair regime), and setting specific waveform characteristics including frequency, amplitude, pulse width, modulation depth, and duty cycle. In some examples, the parameter selection 509 is informed by the patient-specific response model that has been learned over previous treatment sessions, allowing personalized optimization. The adaptive control algorithms may adjust parameter selection based on the severity of the detected event, the patient's current sleep stage, recent stimulation history, and observed effectiveness of previous interventions. According to various embodiments, for predictive interventions where an event is anticipated but not yet occurring, the parameter selection at 509 may choose gentler, preventative stimulation parameters compared to reactive interventions responding to ongoing airway obstruction.

Following parameter selection, the process proceeds to stimulation delivery at 511, where the stimulation module delivers electrical stimulation to the hypoglossal nerve according to the selected parameters. In particular embodiments, the stimulation delivery 511 involves the waveform generation component creating precisely controlled electrical waveforms and the electrode array applying these waveforms to the user's tissue in the region of the hypoglossal nerve. According to various embodiments, the stimulation activates the hypoglossal nerve, causing tongue muscle contraction that opens the airway and restores normal breathing. The stimulation delivery at 511 continues for a duration sufficient to resolve or alleviate the apnea or hypopnea event, with the exact duration determined by ongoing monitoring of the patient's response.

After stimulation delivery at 511, the process proceeds to response monitoring and adjustment of stimulation at 513. In particular embodiments, the response monitoring at 513 involves continuous assessment of the patient's physiological response to the applied stimulation, including monitoring whether airflow has been restored, whether oxygen saturation is improving, whether the apnea event has terminated, and whether the patient shows signs of arousal or discomfort. According to various embodiments, if the initial stimulation parameters are insufficient to resolve the event, stimulation parameters are adjusted in real-time, for example by increasing amplitude, adjusting frequency, changing the active electrode configuration, or switching operational modes. In some examples, if the stimulation successfully resolves the apnea event and normal breathing resumes, the adjustment component of step 513 gradually reduces stimulation intensity or transitions to a maintenance level to prevent immediate recurrence while avoiding over-stimulation. The response monitoring and adjustment at 513 represents the adaptive, closed-loop nature of the system, continuously refining the intervention based on measured outcomes.

From response monitoring and adjustment of stimulation at 513, the process returns to continuous monitoring at 503, completing the closed-loop cycle. According to various embodiments, this return pathway allows the system to continuously assess whether the apnea event has been fully resolved, whether normal breathing is being maintained, and whether any new events are developing. In particular embodiments, the stimulation module continues to stimulate the hypoglossal nerve until apnea events are no longer detected by the monitoring module and the control module, at which point stimulation can be reduced or paused while continuous monitoring 503 continues. This closed-loop architecture ensures that treatment is delivered only when needed and is continuously optimized based on patient response, representing a significant advance over open-loop systems that deliver fixed stimulation patterns regardless of physiological state.

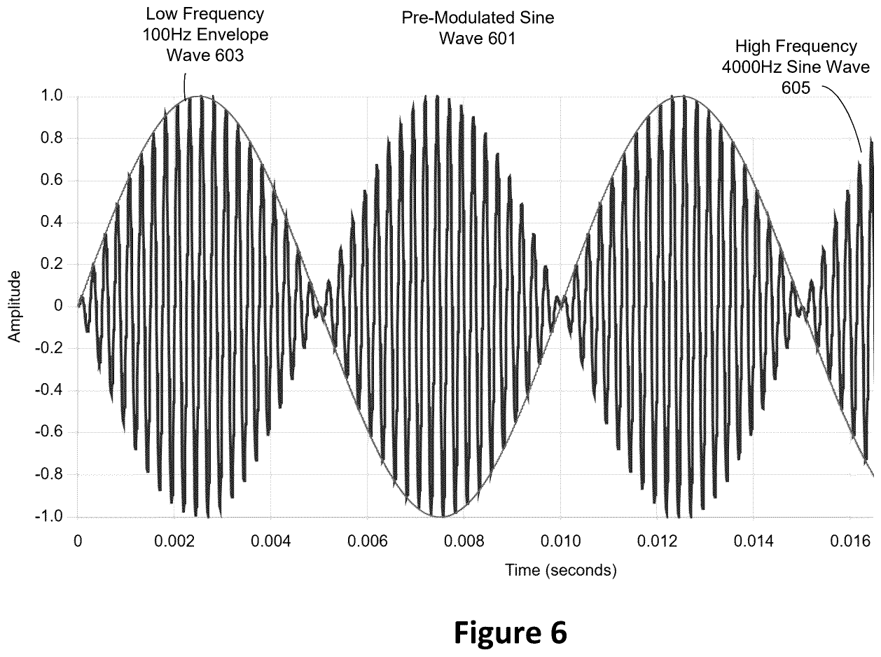

FIG. 6 illustrates an example of a pre-modulated sine wave 601 used in the one-pair electrode regime for hypoglossal nerve stimulation. The pre-modulated sine wave 601 comprises a slowly varying envelope wave 603 with that amplitude-modulates a high-frequency sine wave 605, allowing effective neural stimulation without requiring temporal interference from multiple electrode pairs.

In particular embodiments, the slowly varying wave or slowly varying envelope 603 represents the low-frequency modulation envelope that corresponds to the desired neural activation pattern. According to various embodiments, the slowly varying wave 603 has a frequency in the range of 0-500 Hz, more particularly 10-200 Hz, and in specific implementations 20-100 Hz. In some examples, the slowly varying wave 603 may have a frequency in the range of 20-50 Hz to match the desired hypoglossal nerve neural activation rhythm for maintaining tongue muscle tone and preventing airway collapse during sleep. The slowly varying wave 603 determines the rate at which the hypoglossal nerve fibers are activated, as the nerve membrane responds physiologically to this low-frequency envelope component rather than to the high-frequency carrier.

According to various examples, the high-frequency sine wave 605 serves as the carrier frequency that is amplitude-modulated by the slowly varying wave 603. In particular embodiments, the high-frequency sine wave 605 has a frequency above 1500 Hz, and in particular implementations may be greater than 2000 Hz, 3000 Hz, or 4000 Hz. According to various embodiments, the high-frequency carrier wave 605 provides several important functions including reducing skin impedance through capacitive coupling, minimizing uncomfortable surface sensations that would occur with low-frequency direct stimulation, allowing charge-balanced stimulation with reduced electrochemical reactions at the electrode-tissue interface, and allowing deeper tissue penetration while avoiding activation of superficial cutaneous pain receptors.

In particular embodiments, the composite pre-modulated sine wave 601 shown in FIG. 6 represents the waveform delivered by a single pair of electrodes aligned longitudinally with the hypoglossal nerve. According to various embodiments, the nerve fibers physiologically respond to the slowly varying envelope 603 through a demodulation process inherent in the neural membrane's biophysical properties. The nerve membrane acts as a low-pass filter that is insensitive to the rapid oscillations of the high-frequency carrier 605 but responds to the amplitude variations imposed by the slowly varying envelope 603. As a result, the hypoglossal nerve fibers fire action potentials in synchrony with the slowly varying wave 603, producing muscle contractions at the desired frequency to maintain airway patency. In some examples, the fibers of the hypoglossal nerve demodulate the signal and fire in sync with the low-frequency envelope, allowing for comfortable non-painful stimulation since the high-frequency carrier minimizes activation of cutaneous pain receptors.

According to various embodiments, this pre-modulated waveform approach eliminates the need for temporal interference between multiple electrode pairs while maintaining the therapeutic benefits of high-frequency carrier-based stimulation. In particular embodiments, the waveform shown in FIG. 6 is charge-balanced, meaning that the net charge delivered over each complete cycle is zero or within acceptable safety tolerances, preventing electrochemical tissue damage. The amplitude of both the slowly varying wave 603 and the high-frequency sine wave 605 can be dynamically adjusted by the control module based on real-time physiological feedback, electrode-tissue impedance measurements, and therapeutic efficacy. In some examples, the modulation depth, which represents the extent to which the high-frequency carrier amplitude varies with the envelope, can also be adjusted to optimize the balance between neural activation efficiency and patient comfort.

In particular implementations, while FIG. 6 depicts a sine wave as the basic waveform for both the carrier 605 and envelope 603, alternative embodiments may employ other waveform shapes for the high-frequency carrier including square waves, triangular waves, or other periodic waveforms, provided they maintain characteristics of a high-frequency carrier modulated by a low-frequency envelope. According to various embodiments, the specific choice of carrier frequency, envelope frequency, and waveform shape may be optimized on a patient-specific basis during initial calibration and continuously refined through the adaptive control algorithms in the control module.

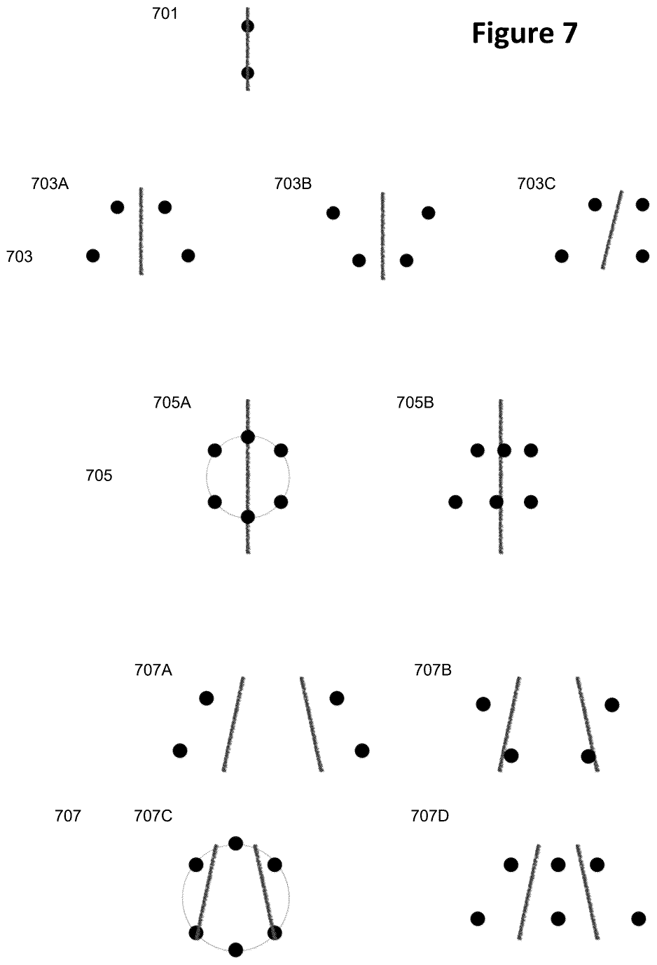

FIG. 7 illustrates examples of various electrode configurations for hypoglossal nerve stimulation to treat sleep apnea. In particular embodiments, configuration 701 depicts a one-pair electrode arrangement for stimulating the hypoglossal nerve on one side of the user's neck. The configuration shows two electrodes (comprising an anode and cathode) aligned longitudinally along the anatomical path of the hypoglossal nerve, represented by the red line. According to various embodiments, this single-pair configuration delivers a pre-modulated biphasic waveform in which a high-frequency carrier wave above 1500 Hz is amplitude-modulated by a low-frequency envelope in the range of 0-500 Hz. The alignment of the electrode pair along the nerve path maximizes the longitudinal electric field gradient, providing the lowest activation threshold and most selective recruitment of hypoglossal nerve fibers. In some examples, this configuration is particularly beneficial for individuals with smaller jaw structures, thinner neck profiles, or anatomies where the hypoglossal nerve is relatively superficial and easily accessible with minimal electrode footprint. The one-pair configuration eliminates the need for temporal interference while maintaining effective neural activation through amplitude modulation, thereby reducing device complexity, cost, and the physical area of electrode coverage required.

Configuration 703A-703C illustrates various two-pair electrode arrangements for stimulating the hypoglossal nerve on one side. In particular embodiments, configuration 703A and 703B show four electrodes arranged in trapezoidal patterns with two pairs positioned at opposite corners, creating a trapezoidal field distribution with the hypoglossal nerve path running through the center. Configuration 703C shows an arrangement where the electrode pairs are positioned at angles relative to the nerve path, creating an oblique field orientation. According to various embodiments, these two-pair configurations allow temporal interference stimulation where each electrode pair delivers a high-frequency biphasic waveform at slightly different frequencies, for example 4000 Hz and 4050 Hz, creating an interference pattern with a beat frequency of 50 Hz at the region where the electric fields overlap. The spatial arrangement of the electrode pairs determines the location and shape of the interference zone, allowing focused stimulation of the hypoglossal nerve at depth while minimizing activation of superficial tissues and cutaneous sensory receptors. In particular implementations, the trapezoidal or geometric arrangements shown in 703 accommodate variations in nerve depth, neck anatomy, and surrounding tissue conductivity by allowing adjustment of field overlap and penetration depth through control of relative amplitudes and frequencies at each electrode pair.

Configuration 705 demonstrates three-pair electrode arrangements for stimulating the hypoglossal nerve on one side with enhanced spatial control. In particular embodiments, configuration 705A shows six electrodes arranged in a circular or hexagonal pattern surrounding the nerve path, with three pairs of electrodes that can be independently controlled. Configuration 705B depicts six electrodes arranged in two parallel rows of three electrodes each, flanking the nerve path. According to various embodiments, the three-pair configurations provide additional degrees of freedom for electric field shaping and current steering compared to one-pair or two-pair arrangements. By independently controlling the phase, amplitude, and frequency of signals delivered by each of the three pairs, the control module can generate more complex interference patterns, improve spatial selectivity to reduce activation of off-target tissues, enhance stimulation efficiency in anatomically complex regions, and adapt to heterogeneous tissue conductivity distributions. In some examples, the three-pair configuration allows dynamic steering of the stimulation focus along or across the nerve path in response to changes in head position, electrode-tissue interface conditions, or therapeutic requirements. The increased number of electrodes in configuration 705 is particularly beneficial for patients with complex anatomy where the hypoglossal nerve path varies significantly from typical anatomical patterns, or where surrounding tissues have unusual conductivity characteristics that require more sophisticated field shaping.

Configuration 707 illustrates electrode arrangements for simultaneous bilateral stimulation of both left and right hypoglossal nerves. In particular embodiments, configuration 707A shows a two-pair arrangement where four electrodes are positioned to target both nerve paths simultaneously, with two electrodes positioned laterally on each side of the neck and the two nerve paths represented by the red lines running between them. Configuration 707B depicts a configuration where the electrode pairs are positioned closer to the midline with reduced lateral spacing, suitable for patients with narrower neck anatomy. Configuration 707C shows a three-pair circular or hexagonal arrangement with six electrodes that can simultaneously stimulate both hypoglossal nerves, providing enhanced field control for bilateral activation. Configuration 707D illustrates a three-pair arrangement with electrodes positioned in two rows, allowing independent or coordinated bilateral stimulation. According to various embodiments, bilateral stimulation may be advantageous for certain patients where unilateral stimulation provides insufficient airway opening, where anatomical asymmetries require balanced activation of both sides, or where therapeutic efficacy is enhanced by coordinated bilateral tongue muscle activation. In some examples, the control module can independently adjust stimulation parameters for the left and right sides to account for asymmetric nerve responsiveness, anatomical variations, or differential therapeutic requirements. The bilateral configurations shown in 707 maintain the flexibility to operate in one-pair, two-pair, or three-pair modes on each side, and can coordinate or independently control stimulation timing, amplitude, and frequency for each nerve.

According to various embodiments, configurations shown in FIG. 7 are implemented using flexible, conformable electrode patches that adhere to the skin surface of the neck and jaw region. In particular embodiments, the electrode placements within these configurations can be customized based on finite element modeling (FEM) simulation derived from patient-specific imaging data such as MRI or CT scans. The control module dynamically selects among these configurations and adjusts active electrodes based on factors including user anatomy, detected apnea severity, electrode-tissue impedance measurements, therapeutic efficacy metrics, and real-time physiological feedback. In some implementations, the system may transition between configurations during a single sleep session in response to changes in sleep position, evolving apnea patterns, or optimization of therapeutic outcomes by the adaptive control algorithms. Although various specific configurations are shown, other configurations such as 4×2, 8×4, 12×6, or other arrangements depending on the coverage area and spatial resolution requirements are possible.

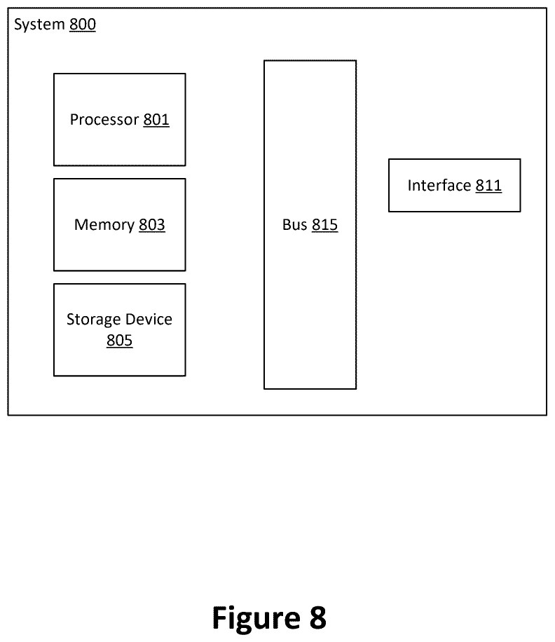

FIG. 8 illustrates one example of a computing device configured in accordance with some embodiments for use in a sleep apnea treatment system. According to various embodiments, system 800 suitable for implementing the control module, monitoring module processing functions, or other computational aspects of the embodiments described herein includes a processor 801, a memory module 803, a storage device 805, an interface 811, and a bus 815 (e.g., a PCI bus or other interconnection fabric). System 800 may operate as a variety of devices such as a dedicated medical device controller, an embedded processor within the stimulation or monitoring modules, a portable computing device for patient data review, or any other computing device or service described herein for sleep apnea treatment and monitoring. Although a particular configuration is described, a variety of alternative configurations are possible.

The processor 801 may perform operations such as those described herein including signal processing, event detection, adaptive control computations, stimulation parameter selection, and safety monitoring. Instructions for performing such operations may be embodied in the memory 803, on one or more non-transitory computer readable media, or on some other storage device. Various specially configured devices can also be used in place of or in addition to the processor 801. The interface 811 may be configured to send and receive data packets over a network for communication with external systems, cloud platforms for population-level analysis, or clinical reporting systems. Examples of supported interfaces include, but are not limited to: Ethernet, fast Ethernet, Gigabit Ethernet, frame relay, cable, digital subscriber line (DSL), token ring, Asynchronous Transfer Mode (ATM), High-Speed Serial Interface (HSSI), and Fiber Distributed Data Interface (FDDI). These interfaces may include ports appropriate for communication with the appropriate media. They may also include an independent processor and/or volatile RAM. A computer system or computing device may include or communicate with a monitor, printer, or other suitable display for providing any of the results mentioned herein to a user, such as displaying sleep quality reports, apnea event summaries, or treatment efficacy data to patients or clinicians.

Any of the disclosed implementations may be embodied in various types of hardware, software, firmware, computer readable media, and combinations thereof. For example, some techniques disclosed herein may be implemented, at least in part, by computer-readable media that include program instructions, state information, etc., for configuring a computing system to perform various services and operations described herein including physiological signal processing, apnea detection, machine learning-based parameter optimization, and closed-loop stimulation control. Examples of program instructions include both machine code, such as produced by a compiler, and higher-level code that may be executed via an interpreter. Instructions may be embodied in any suitable language such as, for example, Apex, Java, Python, C++, C, HTML, any other markup language, JavaScript, ActiveX, VBScript, or Perl. Examples of computer-readable media include, but are not limited to: magnetic media such as hard disks and magnetic tape; optical media such as flash memory, compact disk (CD) or digital versatile disk (DVD); magneto-optical media; and other hardware devices such as read-only memory (“ROM”) devices and random-access memory (“RAM”) devices. A computer-readable medium may be any combination of such storage devices.

What is claimed is:

1. A system, comprising:
a sleep apnea monitoring module having a plurality of physiological sensors configured to track a plurality of physiological parameters associated with a user;
a control module connected to the sleep apnea monitoring module, the control module comprising a processor and configured to detect apnea events associated with the user using the plurality of physiological parameters; and
a stimulation module configured to stimulate a hypoglossal nerve of the user in response to detection of an apnea event, wherein the stimulation module continues to monitor and stimulate the hypoglossal nerve in closed-loop operation until the apnea event is no longer detected by the sleep apnea monitoring module and the control module.

2. The system of claim 1, wherein the stimulation module comprises a single pair of electrodes without requiring additional electrodes for temporal interference based stimulation.

3. The system of claim 2, wherein the single pair of electrodes delivers an electrical signal that includes a high frequency carrier wave amplitude modulated by a low-frequency envelop to the hypoglossal nerve.

4. The system of claim 3, wherein the low-frequency envelope corresponds to a desired hypoglossal nerve neural activation rhythm of 20-50 Hz.

5. The system of claim 4, wherein fibers of the hypoglossal nerve respond to the low-frequency envelope and not rapid oscillations of a high-frequency carrier.

6. The system of claim 5, wherein the fibers of the hypoglossal nerve demodulate the signal and fire in sync with the low-frequency envelope, allowing for comfortable non-painful stimulation since the high-frequency carrier minimizes activation of cutaneous pain receptors.

7. The system of claim 1, wherein a machine learning algorithm is trained on historical physiological data specific to the user to identify patient-specific patterns associated with apnea onset.

8. The system of claim 1, wherein the plurality of physiological sensors comprise electroencephalogram (EEG) sensors.

9. The system of claim 1, wherein the plurality of physiological sensors comprise photoplethysmography (PPG) sensors and functional near-infrared spectroscopy (fNIRS) sensors.

10. The system of claim 1, wherein the plurality of physiological sensors comprise electrocardiogram (ECG) sensors, electromyography (EMG) sensors, and audio sensors.

11. The system of claim 1, wherein the control module dynamically adjusts at least one stimulation parameter selected from the group consisting of: amplitude, frequency, pulse width, electrode configuration, modulation depth, and duty cycle, based on real-time feedback from the monitoring module.

12. The system of claim 1, wherein the system operates in a closed-loop configuration at a control frequency of at least 100 Hz to maintain responsive adaptation to changing physiological conditions.

13. The system of claim 12, wherein the control module automatically selects among operational modes based on at least one factor selected from: user anatomy, detected apnea severity, electrode-tissue impedance measurements, and therapeutic efficacy metrics.

14. The system of claim 1, wherein the stimulation module comprises two pairs of electrodes arranged in a trapezoidal configuration, wherein each pair delivers a high-frequency biphasic waveform with frequencies differing by 10-200 Hz to create a temporal interference pattern at the hypoglossal nerve.

15. The system of claim 14, wherein the high-frequency biphasic waveforms are in a range of 4000-4160 Hz or 10000-10160 Hz.

16. The system of claim 1, wherein the stimulation module comprises three or more pairs of electrodes, and wherein the control module independently controls phase, amplitude, and frequency of signals delivered by each pair to shape an electric field distribution along the hypoglossal nerve.

17. The system of claim 1, further comprising a patient-specific electrode patch that conforms to a user's neck and jawline contours, wherein electrode placement within the patch is customized based on finite element modeling (FEM) simulation derived from imaging data of the user.

18. The system of claim 17, wherein the imaging data comprises at least one of:
magnetic resonance imaging (MRI) data or computed tomography (CT) data of a user's neck and jaw region.

19. A method, comprising:
receiving first monitoring parameters associated with a user;
selecting a first set of electrodes for applying first stimulation parameters based on the first monitoring parameters, wherein the first stimulation parameters include frequency and amplitude, wherein the first set of electrodes are applied to areas of the user corresponding to a hypoglossal nerve;
receiving second monitoring parameters associated with the user; and
selecting a second set of electrodes for applying second stimulation parameters based on the second monitoring parameters, wherein the second stimulation parameters include frequency and amplitude, wherein the second set of electrodes are applied to areas of the user corresponding to the hypoglossal nerve.

20. A method, comprising:
receiving imaging data of a user's neck and jaw region including an area associated with a user's hypoglossal nerve;
creating a model of a user's anatomy based on the imaging data;
designing an electrode array device that conforms to the user's neck and jawline, wherein electrode placement in the electrode array device aligns with the user's hypoglossal nerve;
fabricating the electrode array device, the electrode array device customized to the user and including a plurality of electrodes configured to delivery stimulation to alleviate apnea associated with the user.
